(19) United States
(12) Patent Application Publication
(10) Pub. No.: US 2006/0287233 A1
(43) Pub. Date: Dec. 21, 2006

(54) Method of use of antagonists of zonulin to prevent the loss of or to regenerate pancreatic cells

(76) Inventors: Alessio Fasano, West Friendship, MD (US); Blake Paterson, Baltimore, MD (US); Anna Sapone, Caserta (IT)

Correspondence Address: Connolly Bove Lodge & Hutz LLP;Suite 800, 1990 M Street, N.W., Washington, DC, 20036, US

(21) Appl. No.: 11/449,628

(22) Filed: Jun. 9, 2006

Related U.S. Application Data
(60) Provisional application No. 60/688,693, filed on Jun. 9, 2005.

(51) Int. Cl. A61K 38/22; A61K 38/18

(52) U.S. Cl. 514/12

(57) ABSTRACT

The present invention provides materials and methods for the treatment of diabetes. Using the materials and methods of the invention, the loss of pancreatic β-cells can be slowed and/or prevented. In addition, the materials and methods of the invention can be used to regenerate pancreatic β-cells.

### CROSS REFERENCE TO RELATED APPLICATIONS

This application claims priority to U.S. provisional patent application Ser. No. 60/688,693 filed Jun. 9, 2005, the contents of which are specifically incorporated herein by reference.

### STATEMENT REGARDING FEDERALLY SPONSERED RESEARCH

The development of the present invention was supported by the University of Maryland, Baltimore, Maryland. The invention described herein was supported by funding from the National Institutes of Health (DK 66630 and DK 48373). The Government has certain rights.

### FIELD OF THE INVENTION

The present invention provides materials and methods to prevent or slow the loss of pancreatic β-cells. Further, the present invention also provides materials and methods for regenerating cells, in particular, pancreatic β-cells. In some aspects, antagonists of zonulin (e.g., peptide antagonists) may be used in the practice of the invention.

### BACKGROUND OF THE INVENTION

I. Function and Regulation of Intestinal Tight Junctions

The intestinal epithelium represents the largest interface (more than 2,000,000 cm²) between the external environment and the internal milieu. The maintenance of intercellular tight junctions (“tight junction”) competence prevents movements of potentially harmful environmental factors, such as bacteria, viruses, toxins, food allergens, and macromolecules across the intestinal barrier. This competence is significantly jeopardized in a variety of clinical conditions affecting the gastrointestinal tract, including food allergies, enteric infections, malabsorption syndromes, and inflammatory bowel diseases.

The tj or zonula occludens (hereinafter “ZO”) are one of the hallmarks of absorptive and secretory epithelia (Madara, J. Clin. Invest., 83:1089-1094 (1989); and Madara, Textbook of Secretory Diarrhea, Eds. Lebenthal et al., Chapter 11, pages 125-138 (1990)). As a barrier between apical and basolateral compartments, they selectively regulate the passive diffusion of ions and water-soluble solutes through the paracellular pathway (Gumbiner, Am. J. Physiol., 253 (Cell Physiol. 22):C749-C758 (1987)). This barrier maintains any gradient generated by the activity of pathways associated with the transcellular route (Diamond, Physiologist, 20:10-18 (1977)).

Variations in transepithelial conductance can usually be attributed to changes in the permeability of the paracellular pathway, since the resistances of enterocyte plasma membranes are relatively high (Madara (1989, 1990), supra). The ZO represents the major barrier in this paracellular pathway, and the electrical resistance of epithelial tissues seems to depend on the number of transmembrane protein strands, and their complexity in the ZO, as observed by freeze-fracture electron microscopy (Madara et al., J. Cell Biol. 101:2124-2133 (1985)).

There is abundant evidence that ZO, once regarded as static structures, are in fact dynamic and readily adapt to a variety of developmental (Magnuson et al., Dev. Biol., 67:214-224 (1978); Revel et al., Cold Spring Harbor Symp. Quant. Biol., 40:443-455 (1976); and Schneeberger et al., J. Cell. Sci. 32:307-324 (1978)), physiological (Gilula et al., Dev. Biol., 50:142-168 (1976); Madara et al., J. Membr. Biol., 100:149-164 (1987); Mazariegos et al., J. Cell Biol., 98:1865-1877 (1984); and Sardet et al., J. Cell Biol., 80:96-117 (1979)), and pathological (Milks et al., J. Cell Biol., 103:2729-2738 (1986), Nash et al., Lab. Invest., 59:531-537 (1988); and Shasby et al., Am. J. Physiol., 255 (Cell Physiol., 24:C781-C788 (1988)) circumstances. The regulatory mechanisms that underlie this adaptation are still not completely understood. However, it is clear that, in the presence of Ca²⁺, assembly of the ZO is the result of cellular interactions that trigger a complex cascade of biochemical events that ultimately lead to the formation and modulation of an organized network of ZO elements, the composition of which has been only partially characterized (Diamond, Physiologist, 20:10-18 (1977)). A candidate for the transmembrane protein strands, occluden, has recently been identified (Furuse et al., J. Membr. Biol., 87:141-150 (1985)).

Six proteins have been identified in a cytoplasmic submembranous plaque underlying membrane contacts, but their function remains to be established (Diamond, supra). ZO-1 and ZO-2 exist as a heterodimer (Gumbiner et al., Proc. Natl. Acad. Sci., USA, 88:3460-3464 (1991)) in a detergent-stable complex with an uncharacterized 130 kD protein (ZO-3). Most immunoelectron microscopic studies have localized ZO-1 to precisely beneath membrane contacts (Stevenson et al., Molec. Cell Biochem., 83:129-145 (1988)). Two other proteins, cingulin (Citi et al., Nature (London), 333:272-275 (1988)) and the 7H6 antigen (Zhong et al., J. Cell Biol., 120:477-483 (1993)) are localized further from the membrane and have not yet been cloned. Rab 13, a small GTP binding protein has also recently been localized to the junction region (Zahraoui et al., J. Cell Biol., 124:101-115 (1994)). Other small GTP-binding proteins are known to regulate the cortical cytoskeleton, i.e., rho regulates actin-membrane attachment in focal contacts (Ridley et al., Cell, 70:389-399 (1992)), and rac regulates growth factor-induced membrane ruffling (Ridley et al., Cell, 70:401-410 (1992)). Based on the analogy with the known functions of plaque proteins in the better characterized cell junctions, focal contacts (Guan et al., Nature, 358:690-692 (1992)), and adherens junctions (Tsukita et al., J. Cell Biol., 123:1049-1053 (1993)), it has been hypothesize that tj-associated plaque proteins are involved in transducing signals in both directions across the cell membrane, and in regulating links to the cortical actin cytoskeleton.

To meet the many diverse physiological and pathological challenges to which epithelia are subjected, the ZO must be capable of rapid and coordinated responses that require the presence of a complex regulatory system. The precise characterization of the mechanisms involved in the assembly and regulation of the ZO is an area of current active investigation.

There is now a body of evidence that tj structural and functional linkages exist between the actin cytoskeleton and the tj complex of absorptive cells (Gumbiner et al., supra; Madara et al. supra; and Drenchahn et al., J. Cell Biol., 107:1037-1048 (1988)). The actin cytoskeleton is composed of a complicated meshwork of microfilaments whose precise geometry is regulated by a large cadre of actin-binding proteins. An example of how the state of phosphorylation of an actin-binding protein might regulate cytoskeletal linking to the cell plasma membrane is the myristoylated alanine-rich C kinase substrate (hereinafter “MARCKS”). MARCKS is a specific protein kinase C (hereinafter “PKC”) substrate that is associated with the cytoplasmic face of the plasma membrane (Aderem, Elsevier Sci. Pub. (UK), pages 438-443 (1992)). In its non-phosphorylated form, MARCKS crosslinks to the membrane actin. Thus, it is likely that the actin meshwork associated with the membrane via MARCKS is relatively rigid (Hartwig et al., Nature, 356:618-622 (1992)). Activated PKC phosphorylates MARCKS, which is released from the membrane (Rosen et al., J. Exp. Med., 172:1211-1215 (1990); and Thelen et al., Nature, 351:320-322 (1991)). The actin linked to MARCKS is likely to be spatially separated from the membrane and be more plastic. When MARCKS is dephosphorylated, it returns to the membrane where it once again crosslinks actin (Hartwig et al., supra; and Thelen et al. supra). These data suggest that the F-actin network may be rearranged by a PKC-dependent phosphorylation process that involves actin-binding proteins (MARCKS being one of them).

A variety of intracellular mediators have been shown to alter tj function and/or structure. Tight junctions of amphibian gallbladder (Duffey et al., Nature, 204:451-452 (1981)), and both goldfish (Bakker et al., Am. J. Physiol., 246:G213-G217 (1984)) and flounder (Krasney et al., Fed. Proc., 42:1100 (1983)) intestine, display enhanced resistance to passive ion flow as intracellular cAMP is elevated. Also, exposure of amphibian gallbladder to Ca²⁺ ionophore appears to enhance tj resistance, and induce alterations in tj structure (Palant et al., Am. J. Physiol., 245:C203-C212 (1983)). Further, activation of PKC by phorbol esters increases paracellular permeability both in kidney (Ellis et al., C. Am. J. Physiol., 263 (Renal Fluid Electrolyte Physiol. 32):F293-F300 (1992)), and intestinal (Stenson et al., C. Am. J. Physiol., 265 (Gastrointest. Liver Physiol., 28):G955-G962 (1993)) epithelial cell lines.

II. Zonula Occludens Toxin

Most Vibrio cholerae vaccine candidates constructed by deleting the ctxA gene encoding cholera toxin (CT) are able to elicit high antibody responses, but more than one-half of the vaccinees still develop mild diarrhea (Levine et al., Infect. Immun., 56(1):161-167 (1988)). Given the magnitude of the diarrhea induced in the absence of CT, it was hypothesized that V. cholerae produce other enterotoxigenic factors, which are still present in strains deleted of the ctxA sequence (Levine et al., supra). As a result, a second toxin, zonula occludens toxin (hereinafter “ZOT”) elaborated by V. cholerae and which contribute to the residual diarrhea, was discovered (Fasano et al., Proc. Natl. Acad. Sci., USA, 88:5242-5246 (1991)). The zot gene is located immediately adjacent to the ctx genes. The high percent concurrence of the zot gene with the ctx genes among V. cholerae strains (Johnson et al., J. Clin. Microb., 31(3):732-733 (1993); and

Karasawa et al., FEBS Microbiology Letters, 106:143-146 (1993)) suggests a possible synergistic role of ZOT in the causation of acute dehydrating diarrhea typical of cholera. Recently, the zot gene has also been identified in other enteric pathogens (Tschape, 2nd Asian-Pacific Symposium on Typhoid fever and other Salomellosis, 47 (Abstr.) (1994)).

It has been previously found that, when tested on rabbit ileal mucosa, ZOT increases the intestinal permeability by modulating the structure of intercellular tj (Fasano et al., supra). It has been found that as a consequence of modification of the paracellular pathway, the intestinal mucosa becomes more permeable. It also was found that ZOT does not affect Na⁺-glucose coupled active transport, is not cytotoxic, and fails to completely abolish the transepithelial resistance (Fasano et al., supra).

More recently, it has been found that ZOT is capable of reversibly opening tj in the intestinal mucosa, and thus ZOT, when co-administered with a therapeutic agent, e.g., insulin, is able to effect intestinal delivery of the therapeutic agent, when employed in an oral dosage composition for intestinal drug delivery, e.g., in the treatment of diabetes (WO 96/37196; U.S. Pat. Nos. 5,827,534; 5,665,389; and Fasano et al., J. Clin. Invest., 99:1158-1164 (1997): each of which is incorporated by reference herein in their entirety). It has also been found that ZOT is capable of reversibly opening tj in the nasal mucosa, and thus ZOT, when co-administered with a therapeutic agent, is able to enhance nasal absorption of a therapeutic agent (U. S. Pat. No. 5,908,825; which is incorporated by reference herein in its entirety).

In U.S. Pat. No. 5,864,014; which is incorporated by reference herein in its entirety, a ZOT receptor has been identified and purified from an intestinal cell line, i.e., CaCo2 cells. Further, in U.S. Pat. No. 5,912,323; which is incorporated by reference herein in its entirety, ZOT receptors from human intestinal, heart and brain tissue have been identified and purified. The ZOT receptors represent the first step of the paracellular pathway involved in the regulation of intestinal and nasal permeability.

III. Zonulin

In U.S. Pat. Nos. 5,945,510 and 5,948,629, which are incorporated by reference herein in their entirety, mammalian proteins that are immunologically and functionally related to ZOT, and that function as the physiological modulator of mammalian tight junctions, have been identified and purified. These mammalian proteins, referred to as “zonulin,” are useful for enhancing absorption of therapeutic agents across tj of intestinal and nasal mucosa, as well as across tj of the blood brain barrier.

IV. Peptide Antagonists of Zonulin

Peptide antagonists of zonulin were identified and described for the first time in pending U.S. patent application Ser. No. 09/127,815, filed Aug. 3, 1998, which is incorporated by reference herein in its entirety, which corresponds to WO 00/07609. Peptide antagonists of zonulin may bind to the ZOT receptor, yet not function to physiologically modulate the opening of mammalian tight junctions. The peptide antagonists competitively inhibit the binding of ZOT and zonulin to the ZOT receptor, thereby inhibiting the ability of ZOT and zonulin to physiologically modulate the opening of mammalian tight junctions.

V. Diabetes

Type I diabetes mellitus (T1DM), commonly referred to as insulin-dependent diabetes or juvenile diabetes, is an autoimmune disorder of the pancreas. Patients produce an immune response to β-cells of the pancreas, the cells responsible for the production of insulin. As a result of the destruction of the β-cells, the pancreas can no longer produce the hormone insulin,

The morbidity and mortality associated with diabetes is devastating. The total number of diabetic individuals in the United States is 15.7 million. Of these, 100% of the type I diabetic individuals and 40% of type II diabetic individuals depend on parenteral administration of insulin. On an annual basis, the direct medical costs associated 5 with diabetes exceeds 40 billion dollars. An additional 14 billion dollars is associated with disability, work loss, and premature mortality.

Although oral insulin drug delivery strategies have been the focus of many research efforts, they have been largely unsuccessful because the physiologic nature of the small intestine prevents the absorption of macromolecules, such as insulin.

Recently, United States Patent Publication 2005/0067074 A1 disclosed using peptide antagonists of zonulin to prevent or delay the onset of diabetes. This publication suggests that a critical and early step in disease progression resides in alterations in paracellular permeability and that an increase in paracellular permeability is necessary for the progression toward diabetes. Peptide antagonists of zonulin, which block this endogenous pathway, were shown to prevent the progression to diabetes. Notwithstanding the disclosure of this publication, there remains a need in the art to reverse the course of the disease, for example, by regenerating the insulin-producing β-cells. This need and others are met by the present invention.

### SUMMARY OF THE INVENTION

In some embodiments, the present invention provides a method of slowing the loss of pancreatic β-cells in a subject in need thereof. Such methods may comprise administering to the subject a composition comprising an antagonist of zonulin. An antagonist of zonulin may be a peptide, for example, a peptide comprising the sequence Gly Gly Val Leu Val Gln Pro Gly (SEQ ID NO: 15). Compositions for use in methods of slowing the loss of pancreatic β-cells may comprise one or more components in addition to a zonulin antagonist. For example, compositions may comprise one or more factors that enhance cell growth. Suitable factors include, but are not limited to, growth factors. Examples of suitable growth factors include, but are not limited to, epidermal growth factor (EGF), basic fibroblast growth factor-2 (BFGF-2), keratinocyte growth factor (KGF), hepatocyte growth factor/scatter factor (HGF/SF), glucagon-like-peptide-1 (GLP-1), exendin-4, islet/duodenum homeobox-1 (IDX-1), β-cellulin, activin A, transforming growth factor-α (TGF-α), transforming growth factor-β (TGF-β), gastrin, and combinations thereof.

In some embodiments, the present invention provides a method of regenerating pancreatic β-cells in a subject in need thereof. Such methods may comprise administering to the subject a zonulin antagonist and a cell. An antagonist of zonulin may be a peptide, for example, a peptide comprising the sequence Gly Gly Val Leu Val Gln Pro Gly (SEQ ID NO: 15). Any type of cell that can facilitate the regeneration of β-cells may be used. In some embodiments, the cell may be a cell that secrets growth factors. In some embodiments, the cell may be an islet cell, for example a β-cell. In some embodiments, the cell may be progenitor cell, for example, a stem cell. The timing of the administration of the antagonist and the cell may be optimized using techniques readily known to those of skill in the art. In some embodiments, the antagonist and the cell may be administered simultaneously while in other embodiments, the antagonist and the cell are not administered simultaneously, i.e., the antagonist may be administered before or after the cell is administered. In one embodiment, the antagonist is administered both before and after the cell.

Methods of regenerating pancreatic β-cells in a subject in need thereof comprising administering to the subject a zonulin antagonist and a cell may further comprise administering a factor that enhances cell growth. Suitable factors include, but are not limited to, growth factors. Examples of suitable growth factors include, but are not limited to, epidermal growth factor (EGF), basic fibroblast growth factor-2 (BFGF-2), keratinocyte growth factor (KGF), hepatocyte growth factor/scatter factor (HGF/SF), glucagon-like-peptide-1 (GLP-1), exendin-4, islet/duodenum homeobox-1 (IDX-1), β-cellulin, activin A, transforming growth factor-α (TGF-α), transforming growth factor-β (TGF-β), gastrin, and combinations thereof.

In some embodiments, the present invention provides a method of regenerating pancreatic β-cells in a subject in need thereof, comprising administering to the subject a zonulin antagonist under conditions permitting replication of β-cells. An antagonist of zonulin may be a peptide, for example, a peptide comprising the sequence Gly Gly Val Leu Val Gln Pro Gly (SEQ ID NO: 15). Such methods may further comprise administering a factor that enhances cell growth. Suitable factors include, but are not limited to, growth factors. Examples of suitable growth factors include, but are not limited to, epidermal growth factor (EGF), basic fibroblast growth factor-2 (BFGF-2), keratinocyte growth factor (KGF), hepatocyte growth factor/scatter factor (HGF/SF), glucagon-like-peptide-1 (GLP-1), exendin-4, islet/duodenum homeobox-1 (IDX-1), β-cellulin, activin A, transforming growth factor-α (TGF-α), transforming growth factor-β (TGF-β), gastrin, and combinations thereof.

In some embodiments, the present invention provides a method of regenerating pancreatic β-cells in a subject in need thereof comprising administering to the subject a zonulin antagonist and implanting cells into the subject. An antagonist of zonulin may be a peptide, for example, a peptide comprising the sequence Gly Gly Val Leu Val Gln Pro Gly (SEQ ID NO: 15).

Any type of cells that can be implanted and that facilitate the regeneration of β-cells may be used. In some embodiments, the cells may comprise cells that secret growth factors. In some embodiments, the cells may be islet cells, for example, the cells may comprise β-cells. In some embodiments, the cells may comprise progenitor cells, for example, stem cells. The timing of the administration of the antagonist and implantation of the cells may be optimized using techniques readily known to those of skill in the art. In some embodiments, the antagonist may be administered and the cells implanted simultaneously while in other embodiments, the antagonist is not administered simultaneously with the implantation of the cells, i.e., the antagonist may be administered before or after the cells are implanted. In one embodiment, the antagonist is administered both before and after the cells are implanted.

In some embodiments, a method of regenerating pancreatic β-cells in a subject in need thereof comprising administering to the subject a zonulin antagonist and implanting cells into the subject may further comprise administering a factor that enhances cell growth. Suitable factors include, but are not limited to, growth factors. Examples of suitable growth factors include, but are not limited to, epidermal growth factor (EGF), basic fibroblast growth factor-2 (BFGF-2), keratinocyte growth factor (KGF), hepatocyte growth factor/scatter factor (HGF/SF), glucagon-like-peptide-1 (GLP-1), exendin-4, islet/duodenum homeobox-1 (IDX-1), β-cellulin, activin A, transforming growth factor-α (TGF-α), transforming growth factorβ (TGF-β), gastrin, and combinations thereof. In some embodiments, the factor may be administered and the cells implanted simultaneously while in other embodiments, the factor is not administered simultaneously with the implantation of the cells, i.e., the factor may be administered before or after the cells are implanted. In one embodiment, the factor is administered both before and after the cells are implanted.

The present invention provides a method of treating an autoimmune disease by administering a compound that prevents an increase in permeability of an anatomical barrier. A compound that prevents an increase in the permeability of an anatomical barrier may be an antagonist of a normal physiological compound that increases the permeability of the anatomical barrier. An example of a suitable compound for treatment of autoimmune diseases is a zonulin antagonist. Examples of autoimmune disease that may be treated with a compound that prevents an increase in permeability of an anatomical barrier include, but are not limited to, celiac disease, primary biliary cirrhosis, IgA nephropathy, Wegener's granulomatosis, multiple sclerosis, type 1 diabetes mellitus, rheumatoid arthritis, Crohn's disease, lupus erythematosus, Hashimoto's thyroiditis (underactive thyroid), Graves' disease (overactive thyroid), autoimmune hepatitis, autoimmune inner ear disease, bullous pemphigoid, Devic's syndrome, Goodpasture's syndrome, Lambert-Eaton myasthenic syndrome (LEMS), autoimmune lymphproliferative syndrome (ALPS), paraneoplastic syndromes, polyglandular autoimmune syndromes (PGA), and alopecia areata.

These and other objects of the present invention will be apparent from the detailed description of the invention provided hereinafter.

### BRIEF DESCRIPTION OF THE DRAWINGS

FIG. 1 shows a comparison of the N-terminal sequences of zonulin purified from various human tissues and IgM heavy chain with the N-terminal sequence of the biologically active fragment (amino acids 288-399) of ZOT.

FIG. 2 shows the effect of ZOT, zonulinᵢ, zonulinₕ, either alone (closed bars), or in combination with the peptide antagonist FZI/O (open bars) or in combination with FZI/1 (shaded bars), as compared to the negative control, on the tissue resistance (Rt) of rabbit ileum mounted in Ussing chambers. N equals 3-5;

and * equals p<0.01.

FIG. 3 shows the concentrations (ng/ml) of intraluminal zonulin in both diabetic-prone and diabetic-resistant rats, which was determined using a sandwich ELISA assay. Samples were obtained by intestinal lavage in normal saline. The first bar in each case represents diabetic-resistant rats (DR). The second bar represents diabetic-prone animals (DP), and the third bar represents rats with chronic diabetes (CD). <9% of the diabetic-prone rats do not become diabetic, and ˜9% of the diabetic-resistant rats develop diabetes.

FIG. 4 shows the percentage of rats used in the study that progressed to diabetes.

FIG. 5 shows the concentrations (ng/ml) of intraluminal zonulin in diabetic rats, which was determined using a sandwich ELISA assay.

FIG. 6 shows ex vivo intestinal permeability in diabetic resistant (DR) rats, untreated diabetic-prone rats (DP-untreated; second bar) determined in Ussing chambers, diabetic-prone rats treated with the peptide antagonist of zonulin (DP-treated; third bar). * equals p<0.05; ** equals p<0.05, and p<0.0001 compared to DP-treated.

FIG. 7 shows ex vivo intestinal permeability in the small intestines of untreated diabetes-prone rats that either developed or did not develop diabetes. * equals p<0 4.

FIG. 8 a schematic representation of a model of how aberrant permeability of tight junctions plays a role in the development and progression of Type I diabetes.

FIG. 9 shows haematoxylin and eosin stained sections of pancreata of BBDP rats either untreated or treated with the zonulin inhibitor AT1001. Histological analysis of the pancreata isolated from both untreated rats that developed Type I diabetes (T1D) (top panels) and AT1001-treated rats that did not develop T1D (bottom panels). The islets indicated by the arrows in the left panels (magnification 10×), are shown at higher magnification (40×) in the right panels. Untreated animals revealed end stage islet damage typical of T1D, while treated animals showed evidence of perivascular inflammation without insulitis.

FIG. 10 shows pancreatic islet staining of BBDP rats either untreated or treated with the Zonulin Inhibitor AT1001. Immunohistology of the pancreata isolated from both untreated BBDP rats that developed T1D (top panels) and AT1001-treated rats that did not develop T1D (bottom panels). Islets of rats that developed T1D showed the typical collapsed aspect with no insulin staining (A) and clusters of preserved glucagon-producing delta cells (B). Conversely, AT1001-treated animals showed preserved islets with detectable insulin-producing beta cells (C) at the core of the islet and glucagon-producing delta cells at their edge (D). However, the delta cell staining appeared not uniform and occasionally multiple cell layers (see arrow). Magnification 10×.

FIG. 11 shows immunohistochemistry of the pancreata isolated from both untreated BBDP rats that developed T1D (panels A and B) and AT1001-treated rats that did not develop T1D (panels C-F). Islets from rats that developed T1D showed the typical collapsed aspect with no insulin staining (A) and clusters of preserved glucagon-producing delta cells (B). Conversely, AT1001-treated animals showed islets that were either undamaged (C and D) or showed signs of recovery from an insulitis insult characterized by irregularities in the boundaries between the insulin and glucagon producing cells (E and F). These findings are consistent with the aborting of an ongoing insulitis. Magnification 10×

FIG. 12 shows immunohistochemistry of the pancreata isolated from AT1001-treated rats that did not develop T1D. This islet appeared distorted by intra- and peri-islet scarring that reveals an irregular contour. Sign of recovery from an insulitis insult were visible and were characterized by irregularities in the boundaries between the insulin (A and C) and glucagon (B and D) producing cells (E and F). These findings are consistent with the aborting of an ongoing insulitis.

FIG. 13 shows the results of a study of treatment of autoimmune diabetes with AT-1001. FIG. 13 is a graph is of diabetes free survival plotted as percentage of non-diabetic animals as a function of time comparing untreated animals (•) versus treated animals (▪). BB/wor DP rat were used and therapy was initiated after seroconversion. 60% untreated rats developed T1D, while only 35% of the AT1001-treated animal progressed to T1D. The average age of onset of T1D was 85.4±10.4 days in the placebo group and 86.0±10.3 days in the treated group. The period of the initial study is designated T0, from day 120 on is designated T1.

FIGS. 14A and 14B show the results of a study of treatment of autoimmune diabetes with AT-1001. FIGS. 14A and 14B are bar graphs showing the changes in auto-antibodies during treatment. FIG. 14A shows anti-glutamic acid decarboxylase (GAD) antibodies in animals that developed T1D.

FIG. 14 B shows anti-GAD antibodies in animals that developed T1D.

FIGS. 15A and 15B show the results of a study of treatment of autoimmune diabetes with AT-1001. FIGS. 15A and 15B are bar graphs showing the changes in serum zonulin levels during treatment. FIG. 15A shows zonulin levels in animals that developed T1D. FIG. 15B shows zonulin levels in animals that developed T1D.

### DETAILED DESCRIPTION OF THE INVENTION

As discussed above, in various embodiments, the present invention provides materials and methods for slowing the loss of pancreatic β-cells, preventing the loss of pancreatic β-cells, and/or regenerating pancreatic β-cells in a subject in need thereof by, inter alia, administering to a subject in need of such slowing, preventing and/or regenerating, a pharmaceutically effective amount of an antagonist of zonulin. Typically, antagonists suitable for use in the present invention bind to the zonula occludens toxin (ZOT) receptor, yet do not physiologically modulate the opening of mammalian tight junctions. In some embodiments, the antagonists of zonulin may be peptides. The term “antagonist” is defined as a compound that that prevents, inhibits, reduces or reverses the response triggered by an agonist (i.e., zonulin). In one embodiment, the present invention provides materials and methods for slowing the loss of pancreatic β-cells, preventing the loss of pancreatic β-cells, and/or regenerating pancreatic β-cells in a subject in need thereof by, inter alia, administering to a subject in need of such slowing, preventing and/or regenerating, a pharmaceutically effective amount of an antagonist of zonulin wherein the antagonist binds to the zonula occludens toxin (ZOT) receptor, yet does not physiologically modulate the opening of mammalian tight junctions.

Regenerating pancreatic β-cells as used herein means increasing the number of pancreatic β-cells. Regenerating may entail introducing (e.g., implanting) one or more cells into a subject. Implanting of cells (e.g., β-cells, stem cells, etc) is known in the art. For example, U.S. Pat. No. 6,703,017 (which is specifically incorporated herein by reference particularly Examples 1-3) discloses implanting islet-producing stem cells, islet progenitor cells and islet-like structures. Soon-Shiong, et al. (Proc Natl Acad Sci USA. 90(12):5843-7, (1993)) describe long-term reversal of diabetes by the injection of immunoprotected islets. Isolation of stem cells is known in the art. For example, U.S. Patent Application 20030082155 (which is specifically incorporated herein by reference, particularly Examples 1-4) discloses isolation of stem cells of the islets of langerhans and their use in treating diabetes mellitus. Regenerating pancreatic β-cells may also include providing conditions under which β-cells already present in the pancreas may replicate. For example, it has been shown that adult pancreatic β-cells retain a significant capacity to proliferate in vivo, thus, pancreatic β-cells can be regenerated by providing conditions that facilitate this proliferation. (Dor et al., Nature, 429:41-46 (2002))

As used herein a subject is any animal, e.g., mammal, that receives an antagonist of the invention. Subjects include, but are not limited to, humans.

The present experiments have shown that the development of an autoimmune disease, for example, Type I diabetes, is based on three factors 1) genetic predisposition; 2) leaky anatomical barrier; and 3) repeated environmental insult. Using the materials and methods of the present invention it is possible to treat autoimmune diseases by administering one or more compounds that reduce the permeability of one or more anatomical barriers. As shown below, administration of a compound that antagonizes the activity of the normal physiological compound that enhances the permeability of an anatomical barrier may be used to treat autoimmune diseases. For example, zonulin is a normal physiological compound that enhances the permeability of an anatomical barrier, the gut epithelium. By administering a zonulin antagonist the permeability of an anatomical barrier is maintained or decreased, thereby preventing or treating the autoimmune disease Type I diabetes.

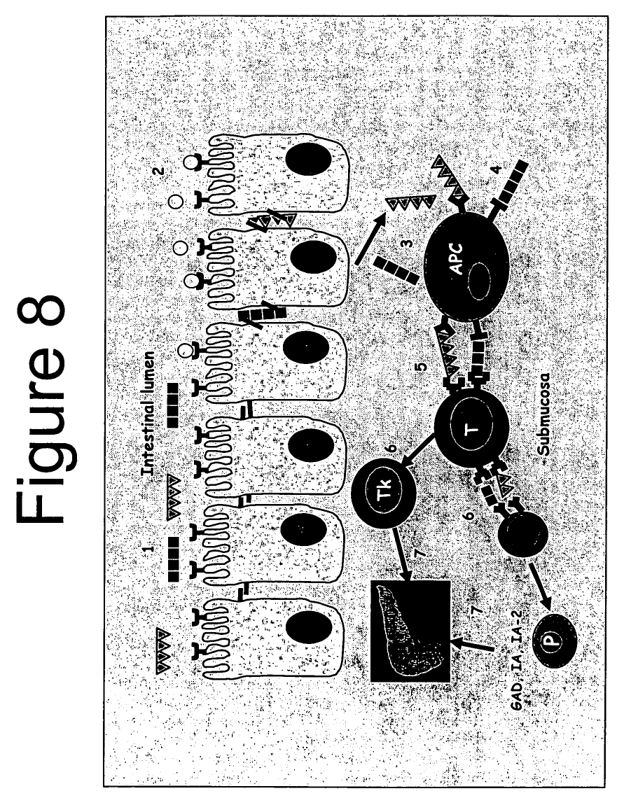

An example of an autoimmune disease in which a leaky anatomical barrier contributes to the development of the disease is Type I diabetes. Without wishing to be bound by theory, it is believed that aberrant intestinal permeability plays a major role in Type 1 diabetes pathogenesis. With reference to FIG. 8, non-self antigens (squares and triangles) are present in the intestinal lumen (1) and cross the tj barriers in subjects with dysregulation of the zonulin system (circles=zonulin, T-shape structures on the cell are zonulin receptors) (2-3). Antigen peptides bind to HLA receptors present on the surface of APC (4). In turn, these peptides are presented to T lymphocytes (5). In genetically susceptible individuals, an aberrant immune response (both humoral and cell-mediated) (6) leads to the autoimmune process mainly targeting the Langherans islets with subsequent insulin deficiency typical of type 1 diabetes (7). Evidence presented below demonstrates that by controlling the permeability of the anatomical barrier, it is possible to reverse the course of the disease and to regenerate the damaged islets.

Thus, the present invention provides a method of treating an autoimmune disease by administering a compound that prevents an increase in permeability of an anatomical barrier. A compound that prevents an increase in the permeability of an anatomical barrier may be an antagonist of a normal physiological compound that increases the permeability of the anatomical barrier. An example of a suitable compound for treatment of autoimmune diseases is a zonulin antagonist.

Any antagonist of zonulin may be used in the practice of the present invention. As used herein an antagonist of zonulin is any compound that bind to the zonulin receptor and that prevents, inhibits, reduces or reverses the response triggered by zonulin. For example, antagonists of the invention may comprise peptide antagonists of zonulin. Examples of peptide antagonists include, but are not limited to, peptides that comprise an amino acid sequence selected from the group consisting of

| | |
|---|---|
| Gly Arg Val Cys Val Gln Pro Gly, | (SEQ ID NO:1) |
| Gly Arg Val Cys Val Gln Asp Gly, | (SEQ ID NO:2) |
| Gly Arg Val Leu Val Gln Pro Gly, | (SEQ ID NO:3) |
| Gly Arg Val Leu Val Gln Asp Gly, | (SEQ ID NO:4) |
| Gly Arg Leu Cys Val Gln Pro Gly, | (SEQ ID NO:5) |
| Gly Arg Leu Cys Val Gln Asp Gly, | (SEQ ID NO:6) |
| Gly Arg Leu Leu Val Gln Pro Gly, | (SEQ ID NO:7) |
| Gly Arg Leu Leu Val Gln Asp Gly, | (SEQ ID NO:8) |
| Gly Arg Gly Cys Val Gln Pro Gly, | (SEQ ID NO:9) |
| Gly Arg Gly Cys Val Gln Asp Gly, | (SEQ ID NO:10) |
| Gly Arg Gly Leu Val Gln Pro Gly, | (SEQ ID NO:11) |
| Gly Arg Gly Leu Val Gln Asp Gly, | (SEQ ID NO:12) |
| Gly Gly Val Cys Val Gln Pro Gly, | (SEQ ID NO:13) |
| Gly Gly Val Cys Val Gln Asp Gly, | (SEQ ID NO:14) |
| Gly Gly Val Leu Val Gln Pro Gly, | (SEQ ID NO:15) |
| Gly Gly Val Leu Val Gln Asp Gly, | (SEQ ID NO:16) |
| Gly Gly Leu Cys Val Gln Pro Gly, | (SEQ ID NO:17) |
| Gly Gly Leu Cys Val Gln Asp Gly, | (SEQ ID NO:18) |
| Gly Gly Leu Leu Val Gln Pro Gly, | (SEQ ID NO:19) |
| Gly Gly Leu Leu Val Gln Asp Gly, | (SEQ ID NO:20) |
| Gly Gly Gly Cys Val Gln Pro Gly, | (SEQ ID NO:21) |
| Gly Gly Gly Cys Val Gln Asp Gly, | (SEQ ID NO:22) |
| Gly Gly Gly Leu Val Gln Pro Gly, | (SEQ ID NO:23) |
| and | |
| Gly Gly Gly Leu Val Gln Asp Gly | (SEQ ID NO:24) |

When the antagonist is a peptide, any length of peptide may be used. Generally, the size of the peptide antagonist will range from about 6 to about 100, from about 6 to about 90, from about 6 to about 80, from about 6 to about 70, from about 6 to about 60, from about 6 to about 50, from about 6 to about 40, from about 6 to about 30, from about 6 to about 25, from about 6 to about 20, from about 6 to about 15, from about 6 to about 14, from about 6 to about 13, from about 6 to about 12, from about 6 to about 11, from about 6 to about 10, from about 6 to about 9, or from about 6 to about 8 amino acids in length. Peptide antagonists of the invention may be from about 8 to about 100, from about 8 to about 90, from about 8 to about 80, from about 8 to about 70, from about 8 to about 60, from about 8 to about 50, from about 8 to about 40, from about 8 to about 30, from about 8 to about 25, from about 8 to about 20, from about 8 to about 15, from about 8 to about 14, from about 8 to about 13, from about 8 to about 12, from about 8 to about 11, or from about 8 to about 10 amino acids in length. Peptide antagonists of the invention may be from about 10 to about 100, from about 10 to about 90, from about 10 to about 80, from about 10 to about 70, from about 10 to about 60, from about 10 to about 50, from about 10 to about 40, from about 10 to about 30, from about 10 to about 25, from about 10 to about 20, from about 10 to about 15, from about 10 to about 14, from about 10 to about 13, or from about 10 to about 12 amino acids in length. Peptide antagonists of the invention may be from about 12 to about 100, from about 12 to about 90, from about 12 to about 80, from about 12 to about 70, from about 12 to about 60, from about 12 to about 50, from about 12 to about 40, from about 12 to about 30, from about 12 to about 25, from about 12 to about 20, from about 12 to about 15, or from about 12 to about 14 amino acids in length. Peptide antagonists of the invention may be from about 15 to about 100, from about 15 to about 90, from about 15 to about 80, from about 15 to about 70, from about 15 to about 60, from about 15 to about 50, from about 15 to about 40, from about 15 to about 30, from about 15 to about 25, from about 15 to about 20, from about 19 to about 15, from about 15 to about 18, or from about 17 to about 15 amino acids in length.

The peptide antagonists can be chemically synthesized and purified using well-known techniques, such as described in High Performance Liquid Chromatography of Peptides and Proteins: Separation Analysis and Conformation, Eds. Mant et al., C.R.C. Press (1991), and a peptide synthesizer, such as Symphony (Protein Technologies, Inc); or by using recombinant DNA techniques, i.e., where the nucleotide sequence encoding the peptide is inserted in an appropriate expression vector, e.g., an E. coli or yeast expression vector, expressed in the respective host cell, and purified therefrom using well-known techniques.

The antagonist (e.g., peptide antagonist)s can be administered as oral dosage compositions for small intestinal delivery. Such oral dosage compositions for small intestinal delivery are well-known in the art, and generally comprise gastroresistent tablets or capsules (Remington's Pharmaceutical Sciences, 16th Ed., Eds. Osol, Mack Publishing Co., Chapter 89 (1980); Digenis et al., J. Pharm. Sci., 83:915-921 (1994); Vantini et al., Clinica Terapeutica, 145:445-451 (1993); Yoshitomi et al., Chem. Pharm. Bull., 40:1902-1905 (1992); Thoma et al., Pharmazie, 46:331-336 (1991); Morishita et al., Drug Design and Delivery, 7:309-319 (1991); and Lin et al., Pharmaceutical Res., 8:919-924 (1991)); each of which is incorporated by reference herein in its entirety). Gastroresistent tablets or capsules of the invention preferably dissolve in intestinal fluids.

Tablets are made gastroresistent by the addition of, e.g., either cellulose acetate phthalate or cellulose acetate terephthalate. The term “gastroresistant” refers to a composition that releases less than 30% by weight of the total zonulin effector in the composition in gastric fluid with a pH of less than 5 or simulated gastric fluid with a pH of less than 5 in sixty minutes.

Capsules are solid dosage forms in which the antagonist (e.g., peptide antagonist) is enclosed in either a hard or soft, soluble container or shell of gelatin. The gelatin used in the manufacture of capsules is obtained from collagenous material by hydrolysis. There are two types of gelatin. Type A, derived from pork skins by acid processing, and Type B, obtained from bones and animal skins by alkaline processing. The use of hard gelatin capsules permit a choice in prescribing a single antagonist (e.g., peptide antagonist) or a combination thereof at the exact dosage level considered best for the individual subject. The hard gelatin capsule consists of two sections, one slipping over the other, thus completely surrounding the antagonist (e.g., peptide antagonist).

These capsules are filled by introducing the antagonist (e.g., peptide antagonist), or gastroresistent beads containing the antagonist (e.g., peptide antagonist), into the longer end of the capsule, and then slipping on the cap. Hard gelatin capsules are made largely from gelatin, FD&C colorants, and sometimes an opacifying agent, such as titanium dioxide. The USP permits the gelatin for this purpose to contain 0.15% (w/v) sulfur dioxide to prevent decomposition during manufacture.

In the context of the present invention, oral dosage compositions for small intestinal delivery also include liquid compositions which contain aqueous buffering agents that prevent the antagonist (e.g., peptide antagonist) from being significantly inactivated by gastric fluids in the stomach, thereby allowing the antagonist (e.g., peptide antagonist) to reach the small intestines in an active form. Examples of such aqueous buffering agents which can be employed in the present invention include bicarbonate buffer (pH 5.5 to 8.7, preferably about pH 7.4).

When the oral dosage composition is a liquid composition, it is preferable that the composition be prepared just prior to administration so as to minimize stability problems. In this case, the liquid composition can be prepared by dissolving lyophilized antagonist (e.g., peptide antagonist) in the aqueous buffering agent.

Typically, compositions comprising a antagonist (e.g., peptide antagonist) of zonulin as used herein comprise a pharmaceutically effective amount of the antagonist. The pharmaceutically effective amount of antagonist (e.g., peptide antagonist) employed may vary according to factors such as the disease state, age, sex, and weight of the individual. Dosage regimens may be adjusted to provide the optimum therapeutic response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

Generally, the amount of an antagonist compound employed in the present invention (for example, to slow the loss of pancreatic β-cells, to prevent the loss of pancreatic β-cells and/or to regenerate pancreatic β-cells) is in the range of about 7.5 μM to 7.5 mM, preferably about 7.5 μM to 0.75 mM. To achieve such a final concentration in, e.g., the intestines or blood, the amount of antagonist (e.g., peptide antagonist) in a single dosage composition of the present invention will generally be from about 50 ng to about 10 μg, from about 250 ng to about 10 μg, from about 500 ng to about 10 μg, from about 1 μg to about 10 μg, from about 2 μg to about 10 μg, from about 3 μg to about 10 μg, from about 4 μg to about 10 μg, from about 5 μg to about 10 μg, from about 50 ng to about 5 μg, from about 250 ng to about 5 μg, from about 500 ng to about 5 μg, from about 1 μg to about 5 μg, from about 2 μg to about 5 μg, from about 3 μg to about 5 μg, from about 4 μg to about 5 μg, from about 50 ng to about 3 μg, from about 250 ng to about 3 μg, from about 500 ng to about 3 μg, from about 1 μg to about 3 μg, or from about 2 μg to about 3 μg per kilogram body weight of the subject.

Compositions of the invention may comprise one or more pharmaceutically-acceptable carriers. As used herein “pharmaceutically-acceptable carrier” includes any and all solvents, dispersion media, coatings, antibacterial and antifungal agents, isotonic and absorption delaying agents, and the like that are physiologically compatible. In one embodiment, the carrier is suitable for parenteral administration. A carrier may be suitable for administration into the central nervous system (e.g., intraspinally or intracerebrally). Alternatively, the carrier can be suitable for intravenous, intraperitoneal or intramuscular administration. In another embodiment, the carrier is suitable for oral administration. Pharmaceutically-acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

The following examples are provided for illustrative purposes only, and are in no way intended to limit the scope of the present invention.

### EXAMPLE 1

Peptide Antagonists of Zonulin

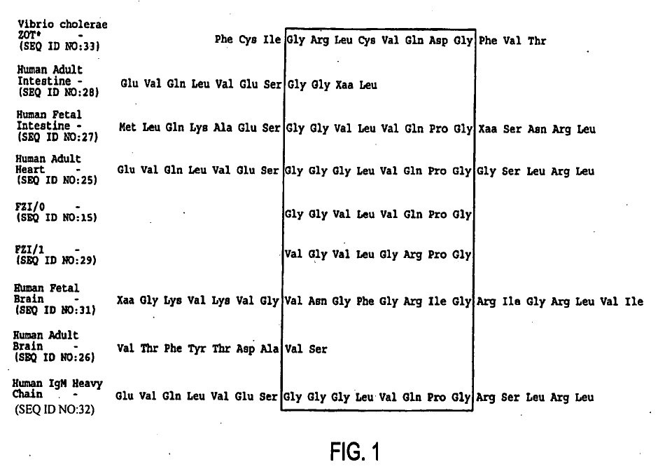

Given that ZOT, human intestinal zonulin (zonulinᵢ) and human heart zonulin (zonulinₕ) all act on intestinal (Fasano et al., Gastroenterology, 112:839 (1997); Fasano et al., J. Clin. Invest. 96:710 (1995), and endothelial tj and that all three have a similar regional effect (Fasano et al., (1997)), that coincides with the ZOT receptor distribution within the intestine (Fasano et al., (1997), supra; and Fasano et al., (1995), supra), it was postulated in U.S. patent application Ser. No. 09/127,815, filed Aug. 3, 1998, that these three molecules interact with the same receptor binding site. A comparison of the primary amino acid structure of ZOT and the human zonulins was thus carried out therein to provide insights as to the absolute structural requirements of the receptor-ligand interaction involved in the regulation of intestinal tj. The analysis of the N-termini of these molecules revealed the following common motif (amino acid residues 8-15 boxed in FIG. 1): non-polar (Gly for intestine, Val for brain), variable, non-polar, variable, non-polar, polar, variable, polar (Gly). Gly in position 8, Val in position 12 and Gln in position 13, all are highly conserved in ZOT, zonulin; and zonulinₕ (see FIG. I), which is believed to be critical for receptor binding function within the intestine. To verify the same, the synthetic octapeptide Gly Gly Val Leu Val Gln Pro Gly (SEQ ID NO: 15) (named FZI/O, and corresponding to amino acid residues 8-15 of human fetal zonulin;) was chemically synthesized.

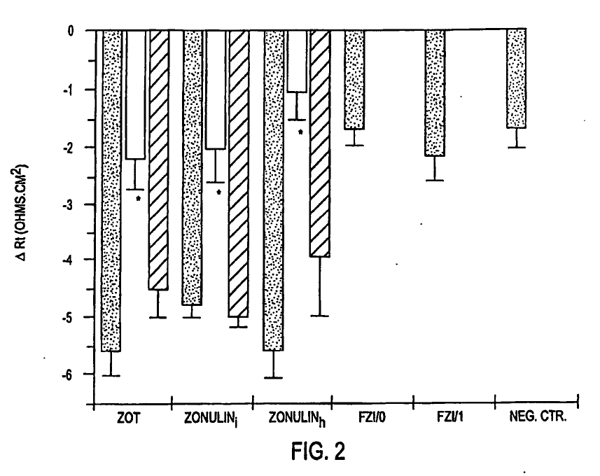

Next, rabbit ileum mounted in Ussing chambers as described below, were exposed to 100 μg of FZI/O (SEQ ID NO:15), 100 μg of FZI/1 (SEQ ID NO:29), 1.0 μg of 6×His-ZOT (obtained as described in Example 1 of U.S. patent application Ser. No. 09/127,815, filed Aug. 3, 1998), 1.0 μg of zonulinᵢ (obtained as described in Example 3 of U.S. patent application Ser. No. 09/127,815, filed Aug. 3, 1998), or 1.0 μg of zonulinₕ (obtained as described in Example 3 of U.S. patent application Ser. No. 09/127,815, filed Aug. 3, 1998), alone; or pre-exposed for 20 min to 100 μg of FZI/O or FZI/1, at which time 1.0 μg of 6×His-ZOT, 1.0 μg of zonulinᵢ, or 1.0 μg of zonulinₕ was added. ΔRt was then calculated as Rt=PD/I_{sc} where PD=potential difference and I_{sc}=short circuit current. The results are shown in FIG. 2.

As shown in FIG. 2, FZI/O did not induce any significant change in Rt (0.5% as compared to the negative control) (see closed bar). On the contrary, pre-treatment for 20 min with FZI/O decreased the effect of ZOT, zonulinᵢ, and zonulinₕ on Rt by 75%, 97%, and 100%, respectively (see open bar). Also as shown in FIG. 2, this inhibitory effect was completely ablated when a second synthetic peptide (FZI/1, SEQ ID NO:29) was chemically synthesized by changing the Gly in position 8, the Val in position 12, and the Gln in position 13 (as referred to zonulinᵢ) with the correspondent amino acid residues of zonulin_{b} (Val, Gly, and Arg, respectively, see SEQ ID NO: 30) was used (see shaded bar). The above results demonstrate that there is a region spanning between residue 8 and 15 of the N-terminal end of ZOT and the zonulin family that is crucial for the binding to the target receptor, and that the amino acid residues in position 8, 12 and 13 determine the tissue specificity of this binding.

### EXAMPLE 2

Diabetic Rat Model

Alterations in intestinal permeability have been shown to be one of the preceding physiologic changes associated with the onset of diabetes (Meddings, Am. J. Physiol., 276:G951-957 (1999)). Paracellular transport and intestinal permeability is regulated by intracellular tj via mechanisms which have not been completely elucidated.

Zonulin and its prokaryotic analog, ZOT, both alter intestinal permeability by modulating tj. In this example, it has been demonstrated for the first time that zonulin-related impairment of tj is involved in the pathogenesis of diabetes, and that diabetes can be prevented, or the onset delayed, by administration of a peptide antagonist of zonulin.

Initially, two genetic breeds, i.e., BB/Wor diabetic-prone (DP) and diabetic-resistant (DR) rats (Haber et al., J. Clin. Invest., 95:832-837 (1993)), were evaluated to determine whether they exhibited significant changes in intraluminal secretion of zonulin and intestinal permeability.

More specifically, age-matched DP and DR rats (20, 50, 75, and >100 days of age) were sacrificed. After the rats were sacrificed, a 25 G needle was placed within the lumen of the ileum, and intestinal lavage with Ringer's solution was performed to determine the presence of intraluminal zonulin. Zonulin concentration was evaluated using a sandwich enzyme linked immunosorbent assay (ELISA) as follows:

Plastic microtiter plates (Costar, Cambridge, Mass.) were coated with polyclonal rabbit anti-ZOT antibodies (obtained as described in Example 2 of U.S. application Ser. No. 09/127,815 filed Aug. 3, 1998) (dilution 1:100) overnight at 4° C., washed three times with PBS containing 0.05% (v/v) Tween 20, then blocked by incubation with 300 μl of PBS containing 0.1% (v/v) Tween 20, for 15 min at room temperature. Next, purified human intestine zonulin (obtained as described in Example 3 of U.S. application Ser. No. 09/127,815 filed Aug. 3, 1998) was coated on the plates.

A standard curve was obtained by diluting zonulin in PBS containing 0.05% (v/v) Tween 20 at different concentration: 0.78 ng/ml, 1.56 ng/ml, 3.125 ng/ml, 6.25 ng/ml, 12.5 ng/ml, 25 ng/ml and 50 ng/ml.

100 μl of each standard concentration or 100 μl of intestinal lavage sample were pipetted into the wells, and incubated for 1 hr at room temperature, using a plate shaker. Unbound zonulin was washed-out using PBS, and the wells were incubated with 100 μl of anti-ZOT antibodies conjugated with alkaline phosphate for 1 hr at room temperature with shaking. Unbound conjugate was washed-out with PBS, and a color reaction was developed by first adding 100 μl of Extra-Avidin (SIGMA, St. Louis, Mo.) diluted 1/20000 in 0.1 M Tris-HCl (pH 7.3), 1.0 mM MgCl₂, 1.0% (w/v) BSA for 15 min, and then incubating each well for 30 min at 37° C. with 100 μl of a solution containing 1.0 mg/ml of p-nitrophenyl-phosphate substrate (SIGMA, St. Louis, Mo.). Absorbance was read on an enzyme immunoassay reader at 405 nm.

In order to evaluate the intra- and inter-assay precision of the ELISA-sandwich method, the coefficient variation (CV) was calculated using three replicates from two samples with different concentrations of zonulin, on three consecutive days. The inter-assay test of the ELISA-sandwich method produced CV values of 9.8%. The CV of the intra-assay test was 4.2% at day 1, 3.3% at day 2 and 2.9% at day 3.

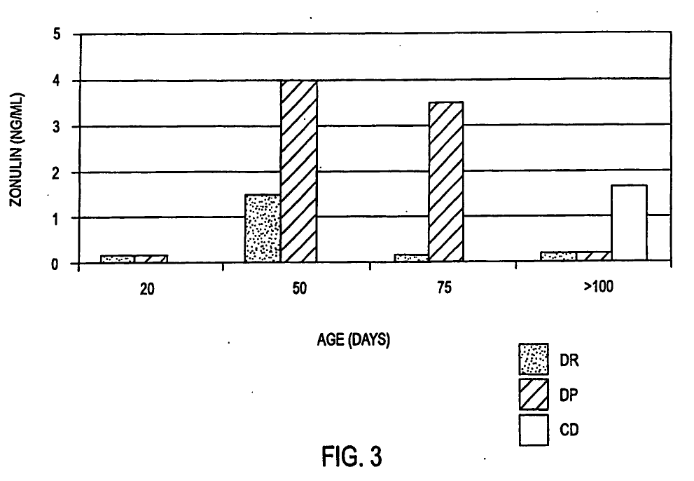

Zonulin concentration was expressed as ng/mg protein detected in the intestinal lavages and normalized by exposed surface area (in mm²). The results are shown in FIG. 3.

As shown in FIG. 3, a 4-fold increase in intraluminal zonulin was first observed in diabetic-prone rats (age 50 days) (second bar). This increase in intraluminal zonulin was found to correlate with an increase in intestinal permeability. The increase in intraluminal zonulin remains high in these diabetic-prone rats, and found to correlate with the progression toward full-blown diabetes. Of note, the diabetic-prone rat (age, 100 days) did not have an increase in intraluminal zonulin. This is remarkable, as this rat did not progress to diabetes. Blood glucose for this rat was normal. Thus, zonulin is responsible for the permeability changes associated with the pathogenesis of type I diabetes. The increase in zonulin secretion is age-related, and proceeds the onset of diabetes.

Next, in order to demonstrate that diabetes can be prevented by administration of a peptide antagonist of zonulin, BB/Wor rats (ages 21-26 days), were obtained from Biomedical Research Models, Inc. (Rutland, Mass.), and were randomized into two groups (n=5 per group), i.e., a treated group and a control group. Both groups were maintained on a standard diet of rat chow (Harlan Teklab Diet #7012). All food and water were previously autoclaved. Each day, daily water intake was measured and 100 ml of fresh water was given. The treated group received 10 μg/ml of the zonulin peptide antagonist (SEQ ID NO: 15) supplemented in the drinking water. The rats were housed in hepa-filter cages.

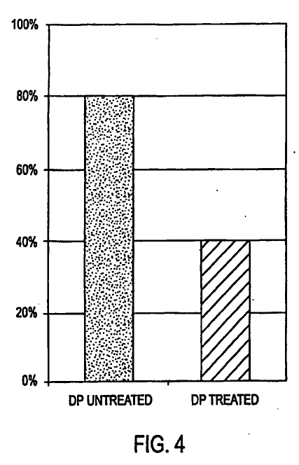

Diabetes in the rats was diagnosed as follows: The rats were weighed twice a week. Blood glucose was determined weekly using the OneTouch® glucose monitoring system (Johnson & Johnson). Each week, reagent strips for urinalysis were used to monitor glucose (Diastix®) and ketones (Ketositx®) (Bayer). Rats with a blood glucose >250 mg/dl were fasted overnight, and blood glucose levels >200 mg/dl were considered diabetic. These guidelines are in accordance with the data supplied by Biomedical Research Models, Inc. The results are shown in FIG. 4.

As shown in FIG. 4, 80% of the control rats (4/5) and 40% of the rats treated with the peptide antagonist of zonulin (2/5) developed diabetes by age 80 days. Alterations in zonulin secretion paralleled the onset of diabetes.

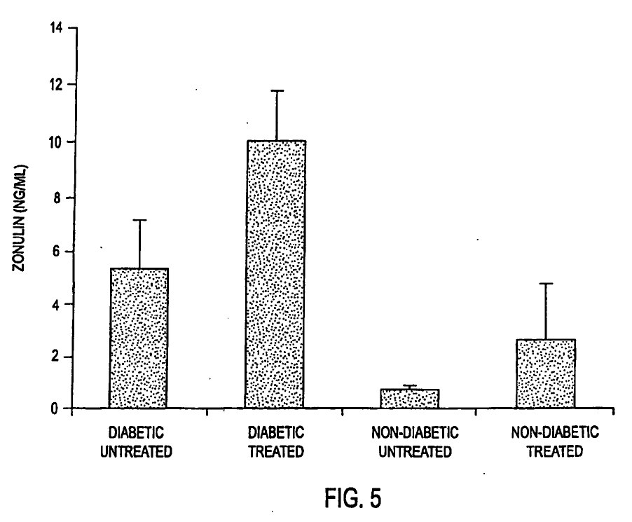

Following clinical presentation of diabetes, the rats were sacrificed as follows: the rats were anesthesized using ketamine anesthesia and a midline incision was made allowing access to the heart. An 18 G needle was placed into the heart and death occurred by exsanguinations. Then, zonulin assays were conducted as described above. For those rats that did not present with diabetes, the endpoint of the study was age 80 days. According to Biomedical Research Models, Inc., 80% of diabetes prone rats present with diabetes by age 80 days. The results of the zonulin assays are shown in FIG. 5.

As shown in FIG. 5, the diabetic rats that were not treated with the peptide antagonist of zonulin were observed to have an increase in intraluminal zonulin, which was consistent with the results shown in FIG. 3. Further, intraluminal zonulin was increased 2 to 4-fold in diabetic rats (DR), as compared to both diabetic-prone rats that did not develop diabetes (DP-treated) and control rats (DP-untreated). Non-diabetic control rats that did not develop diabetes had negligible levels of zonulin, consistent with the levels of zonulin shown in FIG. 3. Moreover, two diabetic-prone rats that developed diabetes despite treatment with the peptide antagonist of zonulin showed intraluminal zonulin levels that were significantly higher than the successfully treated rats, and the untreated control rats. The levels of zonulin were sufficient to initiate the permeability changes necessary to progress to diabetes, but the ZOT/zonulin receptors were effectively blocked by the peptide antagonist.

Also, following clinical presentation of diabetes, the intestinal tissues of the sacrificed rats were mounted in Ussing chamber to assess for changes in ex vivo permeability.

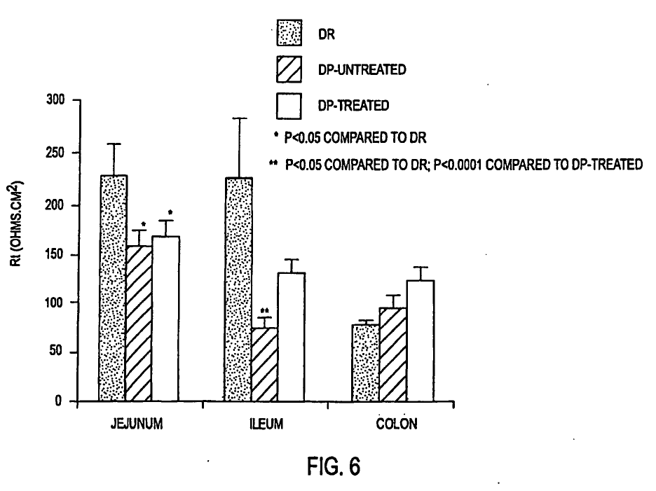

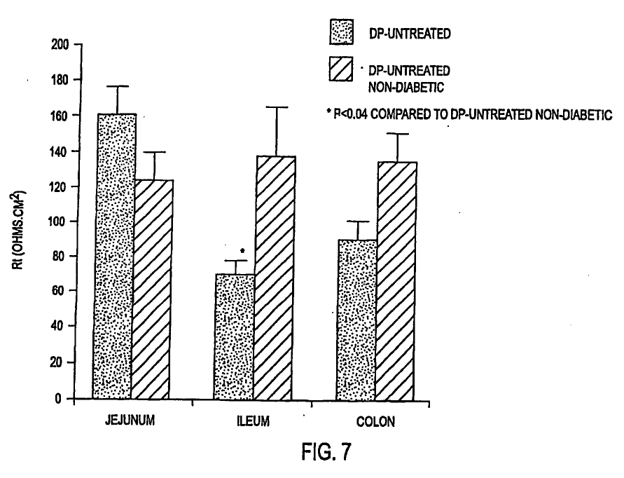

More specifically, sections of jejunum and ileum were isolated from the sacrificed rats, and rinsed free of intestinal contents. Six sections of each intestinal segment was prepared and mounted in Lucite Ussing chambers (0.33 cm² opening), connected to a voltage clamp apparatus (EVC 4000; World Precision Instruments, Saratosa, Fla.), and bathed with freshly prepared buffer comprising 53 mM NaCl, 5.0 mM KCl, 30.5 mM Na₂SO₄, 30.5 mM mannitol, 1.69 mM Na₂P0₄, 0.3 mM NaHP0₄, 1.25 mM CaCl₂, 1.1 mM MgCl₂, and 25 mM NaHC0₃ (pH 7.4). The bathing solution was maintained at 37° C. with water-jacketed reservoirs connected to a constant temperature circulating pump and gassed with 95% O₂ and 5% CO₂. Potential difference was measured and short-circuit current and tissue resistance was calculated as described by Fasano et al., Proc. Natl. Acad. Sci., USA, 88:5242-5246 (1991). The results are shown in FIGS. 6-7.

As demonstrated in the ex vivo Ussing chamber permeability studies, and shown in FIG. 6, all of the rats that progressed to diabetes had an increase in their intestinal permeability. Diabetic resistant (DR) rats had no appreciable alterations in paracellular permeability (first bar). Untreated diabetic-prone rats (DP-untreated; second bar) had a significant increase in paracellular permeability of the jejunum and ileum. More importantly, diabetic-prone rats treated with the peptide antagonist of zonulin (DP-treated; third bar) had a significant increase in paracellular permeability of the small intestine restricted to the jejunum. However, as shown in FIG. 6, pre-treatment with the zonulin peptide antagonist prevented these changes in the distal ileum. Consequently, alterations in paracellular permeability associated with the pathogenesis are restricted to the ileum. Also, as shown in FIG. 6, there are no significant changes in permeability of the colon, which coincides, with the regional distribution of the zonulin receptor distribution.

These results were further validated by a comparison of ex vivo intestinal permeability in the small intestines of untreated diabetes-prone rats that either developed (DP-D) or did not developed (DP-N) diabetes (FIG. 7). While no significant changes in jejunal Rt were observed between DP-D and DP-N rats, a significant lower Rt of the ileal mucosa of DP-D rats was observed as compared to DP-N rats (FIG. 7).

Thus, the following conclusions can be made: (1) the peptide antagonist was able to effectively block the permeability changes required for the development of diabetes; and (2) in those rats treated with the peptide antagonist, the levels of intraluminal zonulin are 3-fold higher than the treated rats that did not develop diabetes. In this population of treated rats that developed diabetes, the amount of peptide antagonist may not have been enough to block a sufficient number of ZOT/zonulin receptors necessary to prevent diabetes.

60% of the treated rats did not develop diabetes. In this population of rats, the peptide antagonist of zonulin effectively prevented the increase in intestinal permeability necessary for the onset of diabetes. As shown in FIG. 5, the treated rats had levels of intraluminal zonulin comparable with the untreated controls, but due to the presence of the peptide antagonist of zonulin, the overall permeability the small intestine was not altered enough to initiate the pathophysiologic changes necessary for the progression to diabetes. Interestingly, as shown in FIG. 5, the one control animal that did not develop diabetes had negligible levels of zonulin, further supporting the role of zonulin in the pathogenesis of diabetes.

Accordingly, an early event in the pathogenesis of diabetes in BB/Wor rats involves changes in zonulin-mediated intestinal paracellular permeability. Furthermore, inhibition of the zonulin signaling system with the use of peptide antagonists of zonulin prevents, or at least delays, the onset of diabetes.

### EXAMPLE 3

Regeneration of β-cells

A test group of 52-54 day old diabetes-prone rats were treated with a zonulin antagonist peptide AT1001 (SEQ ID NO:15) while an age matched control group was not treated. The antagonist was administered at this time because at 40 days these rats show an increase in zonulin levels and at 50 days autoimmune antibodies can be detected. Thus, treatment was started after the onset of diabetes.

BBDP animals age 52-54 were days divided in two groups. Group 1 (n=20) received AT-1001 daily in drinking water+HCO₃. Group 2 (n=10) received drinking water+HCO₃. Animals were randomized to receive either placebo or treatment with the synthetic zonulin peptide inhibitor AT-1001 (SEQ ID:15) in their water supply in a blinded fashion during treatment arm T0. At the disease endpoint (fasting blood glucose>250 mg/dl), BBDP rats that developed T1D (60% incidence in placebo group, average age 110 days) were euthanized and blood and tissue samples collected. AT1001-treated rats that did not develop T1D were re-randomized at age 120 days into 2 groups: a) drug withdrawal arm and b) continued treatment with AT-1001; they were followed for 100 additional days during treatment arm T1. Serum zonulin and autoantibody levels were monitored at the beginning of the study and at its endpoint. Water intake was monitored daily, while weight gain and serum glucose levels were checked weekly. Rats with fasting blood glucose ≧250 mg/dl were considered diabetic and were sacrificed within 24 hours of reaching the diabetic status.

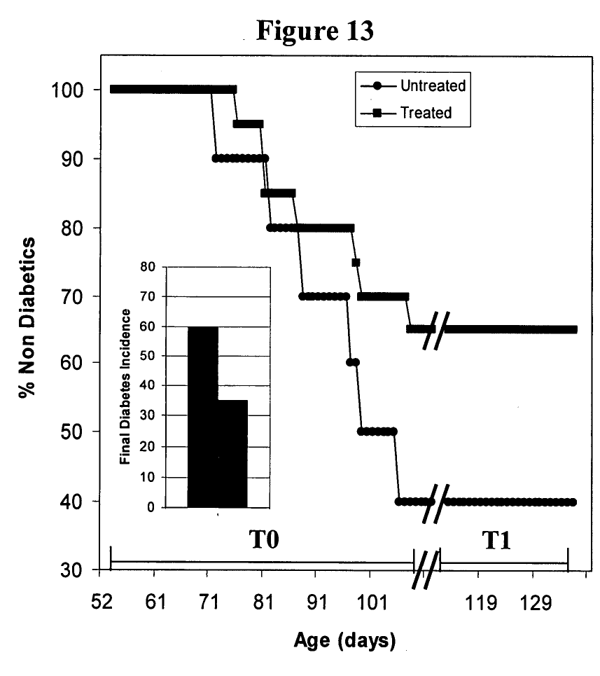

In the untreated control group, 6 of 10 rats developed diabetes while in the treated group only 7 of 20 developed diabetes (FIG. 13). After 120 days, treatment was withdrawn from half of the treated group that had not developed diabetes. ⅓ of the animals from which treatment was withdrawn developed diabetes while none of the animals that continued treatment developed diabetes.

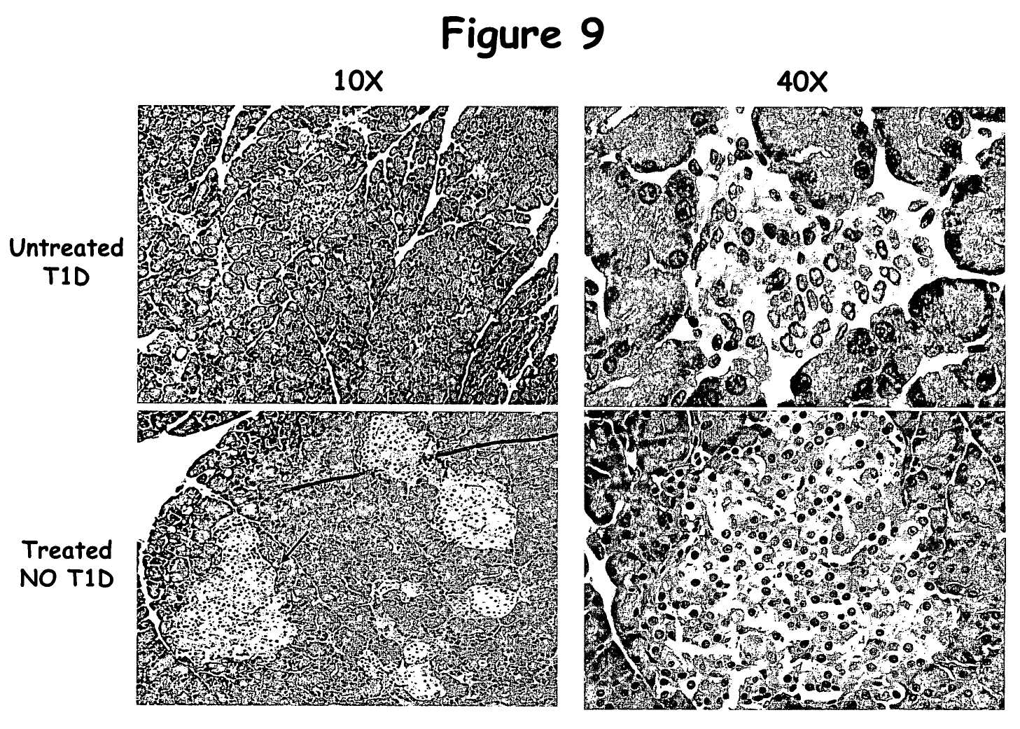

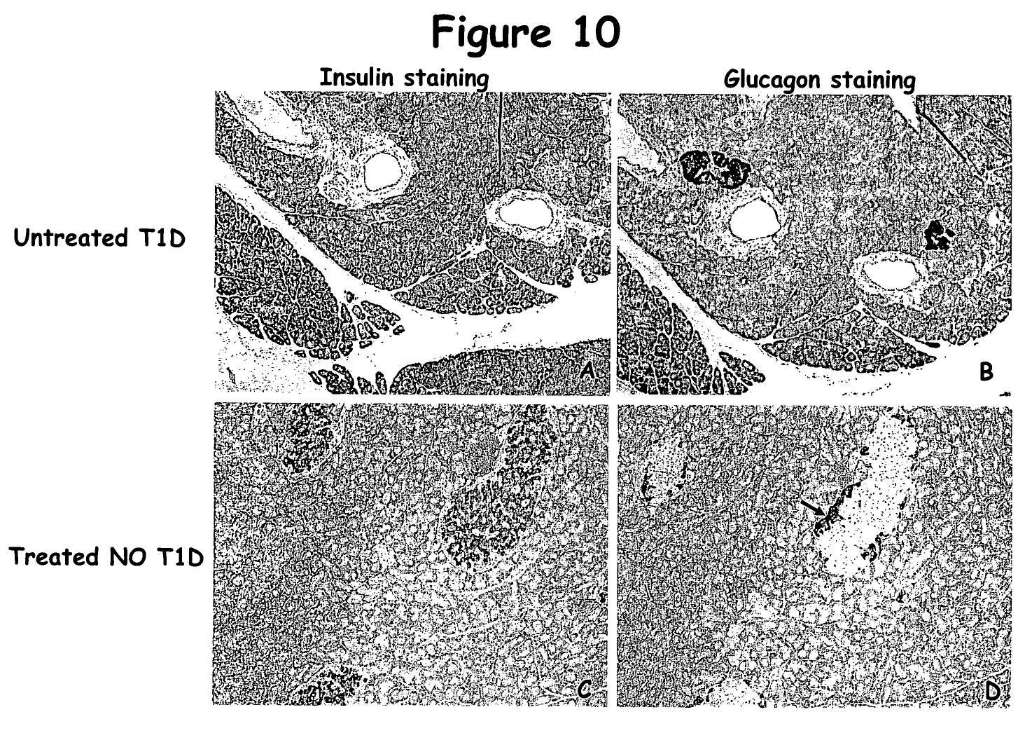

Samples of the pancreas were taken from animals that had been treated starting at day 50-54 and examined. The results of the histological examination are shown in FIG. 9 and the results of the immunohistological examination are shown in FIG. 10. In animals that developed diabetes, histological examination revealed that the β-cells had been destroyed (FIG. 9, top panels). In contrast, samples from animals that did not develop diabetes contained β-cells and evidence of the regeneration of β-cells was observed (FIG. 9, bottom panels).

To verify the identities of the cells observed in the histological analysis, immunohistological examination was conducted. Pancreata were sequentially stained with either anti-glucagon antibody, which is specific for glucon-producing delta cells, or with anti-insulin antibodies, which is specific for insulin-producing β-cells. The results of this analysis are shown in FIG. 10. When untreated pancreas is stained with anti-insulin antibodies, no signal is detected. This is consistent with the destruction of β-cells in T1D. Staining of these cells with anti-glucagon antibodies identifies glucagon producing delta cells. Normal islets have a loaf-shaped structure with the outside of the islet containing delta cells and the inside of the islet containing β-cells. The staining pattern of the delta cells indicates that the islet has collapsed as a result of the destruction of the β-cells (FIG. 10 A & B). In contrast, pancreas from treated animals showed the presence of insulin-producing cells (FIG. 10C). The structure of the islets was more normal as indicated by the staining pattern with anti-glucagon antibodies FIG. 10D.

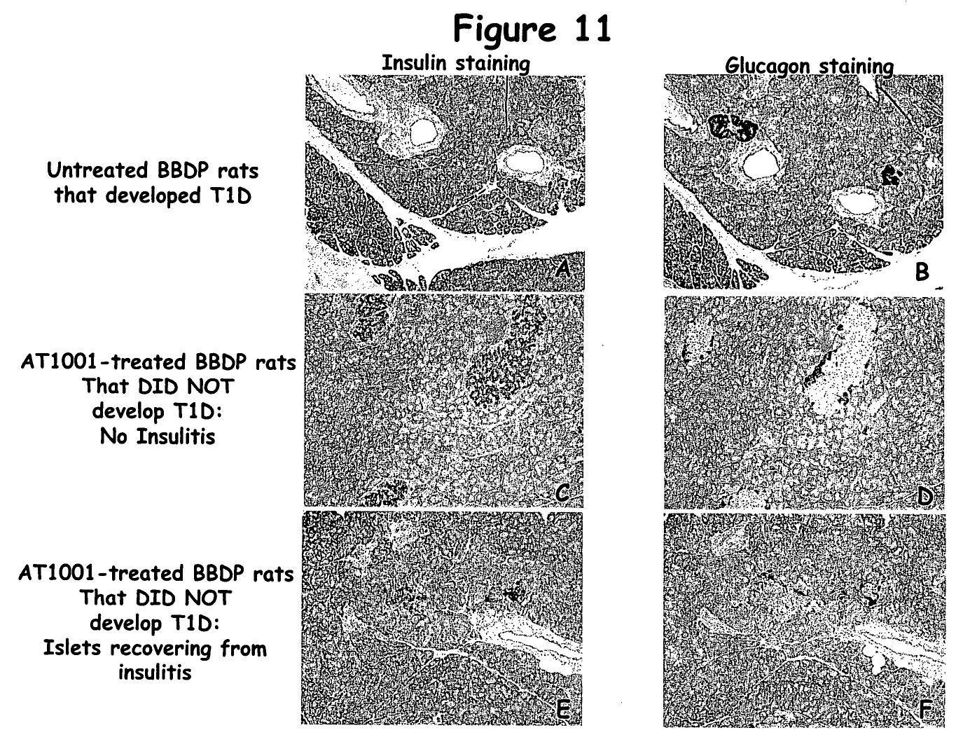

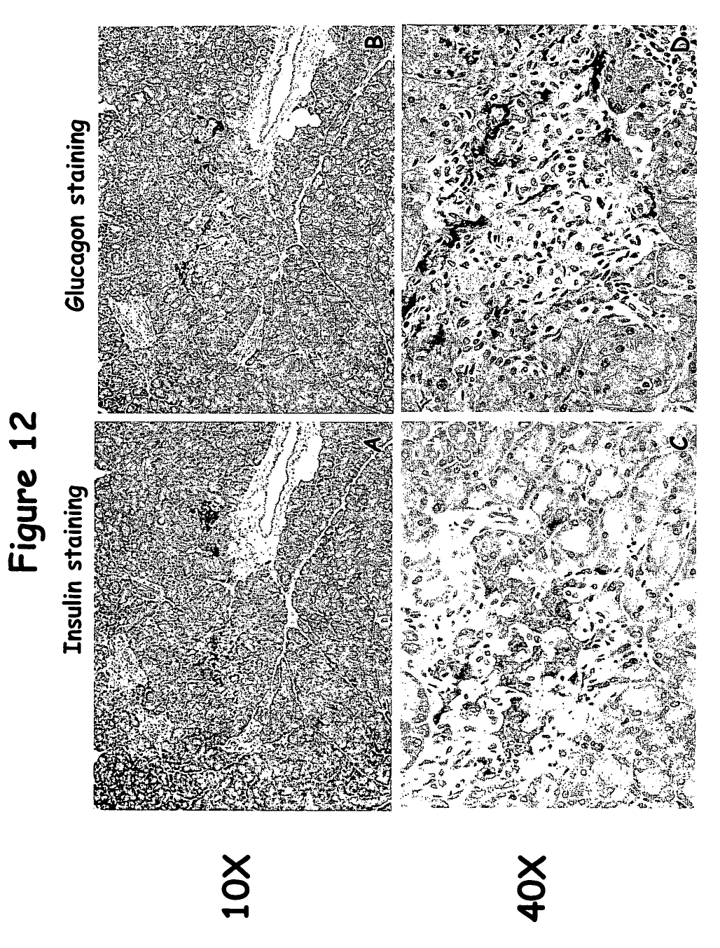

FIGS. 11 and 12 provide evidence of the regeneration of β-cells. FIG. 11 shows the results of immunohistochemical analysis of the pancreata isolated from both untreated BBDP rats that developed T1D (panels A and B) and AT1001-treated rats that did not develop T1D (panels C-F). Islets from rats that developed T1D showed the typical collapsed aspect with no insulin staining (A) and clusters of preserved glucagon-producing delta cells (B). Conversely, AT1001-treated animals showed islets that were either undamaged (C and D) or showed signs of recovery from an insulitis insult characterized by irregularities in the boundaries between the insulin and glucagon producing cells (E and F). FIG. 12 shows higher magnification of panels 11E and 11F. The infiltration of the delta cells into the islet (FIG. 12D) occurs as a result of the regeneration of the β-cells after insult.

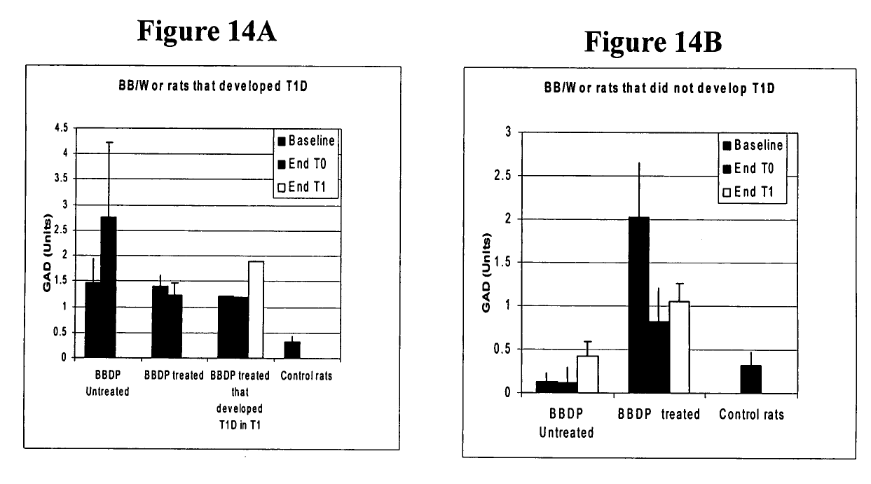

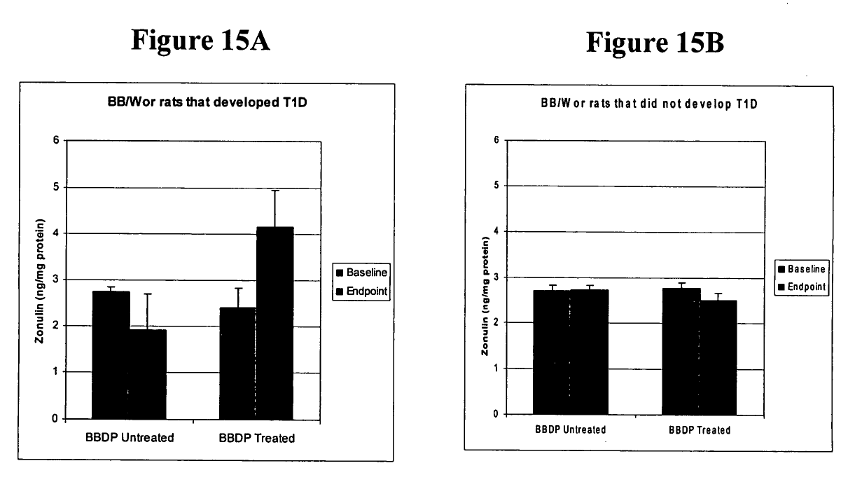

The blockage of the zonulin pathway in BBDP rats at their preclinical autoimmune stage significantly reduced the progression to T1D up to age 205 days (150 days post-treatment). This decreased incidence of T1D was associated to a significant reduction of the anti-glutamic acid decarboxylase (GAD) antibodies following AT1001 treatment (FIG. 14). AT1001 treatment did not affect serum zonulin levels during the study (FIG. 15). Withdraw of AT1001 treatment was followed by onset of T1D in 33% of the animals. AT1001-treated BBDP rats showed either normal islet histology or islet showing signs of recovery from insulitis as compared to untreated rats that showed end stage islet damage typical of T1D. Combined together, these data suggest that AT1001 was able to abort and revert the islet insult in BBDP rats even if the autoimmune process is already started.

While the invention has been described in detail, and with reference to specific embodiments thereof, it will be apparent to one of ordinary skill in the art that various changes and modifications can be made therein without departing from the spirit and scope thereof.

What is claimed is:

1. A method of slowing the loss of pancreatic β-cells in a subject in need thereof, comprising:
administering to the subject a composition comprising an antagonist of zonulin.

2. A method according to claim 1, wherein the composition further comprises a factor that enhances cell growth.

3. A method according to claim 2, wherein the factor is a growth factor.

4. A method according to claim 2, wherein the factor is selected from a group consisting of epidermal growth factor (EGF), basic fibroblast growth factor-2 (BFGF-2), keratinocyte growth factor (KGF), hepatocyte growth factor/scatter factor (HGF/SF), glucagon-like-peptide-1 (GLP-1), exendin-4, islet/duodenum homeobox-1 (IDX-1), β-cellulin, activin A, transforming growth factor-α (TGF-α), transforming growth factor-β (TGF-β), gastrin, and combinations thereof.

5. A method of regenerating pancreatic β-cells in a subject in need thereof, comprising:
administering to the subject a zonulin antagonist and a cell.

6. A method according to claim 5, wherein the cell is an islet cell.

7. A method according to claim 5, wherein the cell is a β-cell.

8. A method according to claim 5, wherein the cell is a stem cell.

9. A method according to claim 5, wherein the antagonist and the cell are administered simultaneously.

10. A method according to claim 5, wherein the antagonist and the cell are not administered simultaneously.

11. A method according to claim 5, further comprising administering a factor that enhances cell growth.

12. A method according to claim 11, wherein the factor is a growth factor.

13. A method according to claim 12, wherein the factor is selected from a group consisting of epidermal growth factor (EGF), basic fibroblast growth factor-2 (BFGF-2), keratinocyte growth factor (KGF), hepatocyte growth factor/scatter factor (HGF/SF), glucagon-like-peptide-1 (GLP-1), exendin-4, islet/duodenum homeobox-1 (IDX-1), β-cellulin, activin A, transforming growth factor-α (TGF-α), transforming growth factor-β (TGF-β), gastrin, and combinations thereof.

14. A method of regenerating pancreatic β-cells in a subject in need thereof, comprising:
administering to the subject a zonulin antagonist under conditions permitting replication of β-cells.

15. A method according to claim 14, further comprising administering a factor that enhances cell growth.

16. A method according to claim 14, wherein the factor is a growth factor.

17. A method according to claim 14, wherein the factor is selected from a group consisting of epidermal growth factor (EGF), basic fibroblast growth factor-2 (BFGF-2), keratinocyte growth factor (KGF), hepatocyte growth factor/scatter factor (HGF/SF), glucagon-like-peptide-1 (GLP-1), exendin-4, islet/duodenum homeobox-1 (IDX-1), β-cellulin, activin A, transforming growth factor-α (TGF-α), transforming growth factor-β (TGF-β), gastrin, and combinations thereof.

18. A method of regenerating pancreatic β-cells in a subject in need thereof, comprising:
administering to the subject a zonulin antagonist; and
implanting cells into the subject.

19. A method according to claim 18, wherein the cells are islet cells.

20. A method according to claim 18, wherein the cells are β-cells.

21. A method according to claim 18, wherein the cells are stem cells.

22. A method according to claim 18, wherein the antagonist is administered to the subject before the cells are implanted.

23. A method according to claim 18, wherein the antagonist is administered to the subject after the cells are implanted.

24. A method according to claim 18, wherein the antagonist is administered to the subject both before and after the cells are implanted.

25. A method according to claim 18, further comprising administering a factor that enhances cell growth.

26. A method according to claim 18, wherein the factor is a growth factor.

27. A method according to claim 18, wherein the factor is selected from a group consisting of epidermal growth factor (EGF), basic fibroblast growth factor-2 (BFGF-2), keratinocyte growth factor (KGF), hepatocyte growth factor/scatter factor (HGF/SF), glucagon-like-peptide-1 (GLP-1), exendin-4, islet/duodenum homeobox-1 (IDX-1), β-cellulin, activin A, transforming growth factor-α (TGF-α), transforming growth factor-β (TGF-β), gastrin, and combinations thereof.

28. A method according to claim 25, wherein the factor is administered to the subject before the cells are implanted.

29. A method according to claim 25, wherein the factor is administered to the subject after the cells are implanted.

30. A method according to claim 25, wherein the factor is administered to the subject both before and after the cells are implanted.

31. A method of treating an autoimmune disease, comprising:
administering a compound that prevents an increase in permeability of an anatomical barrier.

32. The method of claim 31, wherein the compound that prevents an increase in the permeability of an anatomical barrier is an antagonist of a normal physiological compound that increases the permeability of the anatomical barrier.

33. The method of claim 31, wherein the compound is a zonulin antagonist.

34. The method of claim 33, wherein the zonulin antagonist comprises SEQ ID NO:15.

35. The method of claim 31, wherein the compound is selected from the group consisting of SEQ IDs 1-24.


SEQUENCE LISTING (33 sequences)

SEQ ID NO: 1 - protein, 8 aa, Artificial
  sythetic peptide
GRVCVQPG

SEQ ID NO: 2 - protein, 8 aa, Artificial
  sythetic peptide
GRVCVQDG

SEQ ID NO: 3 - protein, 8 aa, Artificial
  sythetic peptide
GRVLVQPG

SEQ ID NO: 4 - protein, 8 aa, Artificial
  sythetic peptide
GRVLVQDG

SEQ ID NO: 5 - protein, 8 aa, Artificial
  sythetic peptide
GRLCVQPG

SEQ ID NO: 6 - protein, 8 aa, Artificial
  sythetic peptide
GRLCVQDG

SEQ ID NO: 7 - protein, 8 aa, Artificial
  sythetic peptide
GRLLVQPG

SEQ ID NO: 8 - protein, 8 aa, Artificial
  sythetic peptide
GRLLVQDG

SEQ ID NO: 9 - protein, 8 aa, Artificial
  sythetic peptide
GRGCVQPG

SEQ ID NO: 10 - protein, 8 aa, Artificial
  sythetic peptide
GRGCVQDG

SEQ ID NO: 11 - protein, 8 aa, Artificial
  sythetic peptide
GRGLVQPG

SEQ ID NO: 12 - protein, 8 aa, Artificial
  sythetic peptide
GRGLVQDG

SEQ ID NO: 13 - protein, 8 aa, Artificial
  sythetic peptide
GGVCVQPG

SEQ ID NO: 14 - protein, 8 aa, Artificial
  sythetic peptide
GGVCVQDG

SEQ ID NO: 15 - protein, 8 aa, Artificial
  sythetic peptide
GGVLVQPG

SEQ ID NO: 16 - protein, 8 aa, Artificial
  sythetic peptide
GGVLVQDG

SEQ ID NO: 17 - protein, 8 aa, Artificial
  sythetic peptide
GGLCVQPG

SEQ ID NO: 18 - protein, 8 aa, Artificial
  sythetic peptide
GGLCVQDG

SEQ ID NO: 19 - protein, 8 aa, Artificial
  sythetic peptide
GGLLVQPG

SEQ ID NO: 20 - protein, 8 aa, Artificial
  sythetic peptide
GGLLVQDG

SEQ ID NO: 21 - protein, 8 aa, Artificial
  sythetic peptide
GGGCVQPG

SEQ ID NO: 22 - protein, 8 aa, Artificial
  sythetic peptide
GGGCVQDG

SEQ ID NO: 23 - protein, 8 aa, Artificial
  sythetic peptide
GGGLVQPG

SEQ ID NO: 24 - protein, 8 aa, Artificial
  sythetic peptide
GGGLVQDG

SEQ ID NO: 25 - protein, 20 aa, Homo sapiens
EVQLVESGGGLVQPGGSLRL

SEQ ID NO: 26 - protein, 9 aa, Homo sapiens
VTFYTDAVS

SEQ ID NO: 27 - protein, 20 aa, Homo sapiens
  misc_feature (16)..(16): Xaa can be any naturally occurring amino acid
MLQKAESGGVLVQPGXSNRL

SEQ ID NO: 28 - protein, 11 aa, Homo sapiens
  misc_feature (10)..(10): Xaa can be any naturally occurring amino acid
EVQLVESGGXL

SEQ ID NO: 29 - protein, 8 aa, Artificial
  sythetic peptide
VGVLGRPG

SEQ ID NO: 30 - protein, 8 aa, Artificial
  sythetic peptide
VDGFGRIG

SEQ ID NO: 31 - protein, 22 aa, Homo sapiens
  misc_feature (1)..(1): Xaa can be any naturally occurring amino acid
XGKVKVGVNGFGRIGRIGRLVI

SEQ ID NO: 32 - protein, 20 aa, Homo sapiens
EVQLVESGGGLVQPGRSLRL

SEQ ID NO: 33 - protein, 13 aa, Vibrio cholerae
FCIGRLCVQDGFV